(19) United States
(12) Patent Application Publication
(10) Pub. No.: US 2019/0231290 A1
(43) Pub. Date: Aug. 1, 2019

(54) MAMMOGRAPHY-APPARATUS AND METHOD OF MEASURING A CONTACT AREA BETWEEN A BREAST AND THE MAMMOGRAPHY-APPARATUS

(71) Applicant: Sigmascreening B.V., Enschede (NL)

(72) Inventors: Gerardus Johannes den Heeten, Enschede (NL); Cornelis Antonius Grimbergen, Enschede (NL)

(21) Appl. No.: 16/376,691

(22) Filed: Apr. 5, 2019

Related U.S. Application Data
(63) Continuation of application No. PCT/NL2017/050653, filed on Oct. 3, 2017.

(30) Foreign Application Priority Data
Oct. 5, 2016 (NL) 2017578

(51) Int. Cl. A61B 6/00; A61B 5/00; A61B 6/04

(52) U.S. Cl. CPC A61B 6/502; A61B 5/0053; A61B 6/0414; A61B 5/0091; A61B 5/1075; A61B 5/0064; A61B 2576/02; A61B 2562/164; A61B 2560/0406; A61B 5/708

(57) ABSTRACT

Mammography-apparatus (1) for detecting malignant cells in a breast (2) comprising an x-ray source (3) and an x-ray detector (4′) that cooperates with the x-ray source (3) for providing an x-ray image of said breast (2), and further comprising a paddle (5) for flattening the breast (2) by pressing it against an x-ray detector cover (4), and comprising a contact area measuring device for measuring a contact area between the breast (2) and the mammography-apparatus (1), wherein the paddle (5) is replaceable and the contact area measuring device is only provided on or incorporated in the detector cover (4) facing the breast (2) and the paddle (5) is embodied without a contact area measuring device.

### CROSS-REFERENCE TO RELATED APPLICATIONS

This application is a continuation of International Patent Application No. PCT/NL2017/050653 titled “MAMMOGRAPHY-APPARATUS AND METHOD OF MEASURING A CONTACT AREA BETWEEN A BREAST AND THE MAMMOGRAPHY-APPARATUS”, filed on Oct. 3, 2017, which claims priority to Netherlands Patent Application No. 2017578 filed on Oct. 5, 2016, and the specification and claims thereof are incorporated herein by reference.

### STATEMENT REGARDING FEDERALLY SPONSORED RESEARCH OR DEVELOPMENT

Not Applicable.

### THE NAMES OF PARTIES TO A JOINT RESEARCH AGREEMENT

Not Applicable.

### INCORPORATION BY REFERENCE OF MATERIAL SUBMITTED ON A COMPACT DISC

Not Applicable.

### STATEMENT REGARDING PRIOR DISCLOSURES BY THE INVENTOR OR A JOINT INVENTOR

Not Applicable.

### COPYRIGHTED MATERIAL

Not Applicable.

### BACKGROUND

The invention relates to a mammography-apparatus for detecting malignant cells in a breast, which apparatus comprises an x-ray source and an x-ray detector that cooperates with the x-ray source for providing an x-ray image of said breast, and further comprises a paddle for flattening the breast by pressing it against said x-ray detector.

Such a mammography-apparatus is known from US2008/0240346 in which a pressure detector is applied for detecting pressures applied by a compression plate to an image capturing base, and wherein the pressure detector may comprise a pressure sensitive sheet.

US2012/0020455 teaches a mammography-apparatus for detecting malignant cells in a breast comprising an x-ray source and an x-ray detector that cooperates with the x-ray source for providing an x-ray image of said breast, and further comprising a paddle for flattening the breast by pressing it against an x-ray detector cover, and comprising contact area measuring sensors for measuring a contact area between the breast and the mammography-apparatus, wherein the sensors are mounted in the detector surface or at the underside of the paddle.

In US2007/0121782 the size of a breast is determined according to the size of the compression paddle, or by pre-exposing the breast with a small amount of radiation by the breast size detection means prior to imaging. A solid-state detector which is used for the pre-exposure will have different pixel values between the breast area and the area directly exposed by the radiation. Alternatively a CCD camera may be used, or a strain gauge provided in the object table, or temperature sensors provided on the side of the object table facing the chest.

In US2008/0043904 a compression plate comprising elastically deformable elements is used, wherein an adaptation of the shape of the individual compression plate elements is achieved to the anatomy of the female breast.

A mammography-apparatus according to the preamble is further known from WO2011/102713 and comprises a contact area measuring device employing an optical system for measuring a contact area between the breast and the paddle.

According to WO2011/102713 the contact area measuring device can be used in a dual fashion. In an embodiment in which the force that is applied to the breast is measured, this force together with the contact area provides an estimation of the average pressure that is applied to the breast. This average pressure can then be controlled at a pre-established level so as to avoid unnecessary and avoidable pain during imaging.

Further, the measured contact area between the breast and the paddle resulting from the breast-compression can be used together with the pre-established force-level, to calculate and apply a specific mean compression pressure independent of the dimensions of the individual breast. Knowing and controlling this specific mean compression pressure leads to a better standardization of the mammography operation, with improved accuracy of screening whilst avoiding unnecessary pain for the persons being screened.

US 2014/341338 which corresponds to EP 2 819 581 teaches that the contact area measuring device can be embodied with at least a first laminate of an electrically insulating material and an electrically low resistance material, which first laminate is provided on a side of the paddle facing the breast, wherein the electrically low resistance material is sandwiched between the paddle and the insulating material. This opens the way to carry out an electrical measurement of the actual area at which the breast and paddle are in contact with each other. According to US 2014/341338 (and EP 2 819 581) the measuring of the contact area between the breast and the paddle of the mammography-apparatus that further comprises an x-ray detector with a cover, is then carried out such that the electrically conducting x-ray detector cover and the electrically low resistance material of the first laminate are connected to a measurement circuit which is powered by an alternating current excitation source, wherein the electrical potential is measured between the x-ray detector and the electrically low resistance material of the first laminate on the paddle, and that said electrical potential is used as a measure for the contact area between the breast and the paddle. This measurement of the contact area is then based on the change of capacitance between the x-ray detector cover and the electrically low resistance material of the first laminate on the paddle, as determined by the breast placed between the paddle and the x-ray detector cover.

US 2014/341338 which corresponds to EP 2 819 581 further discloses that it may be beneficial that the contact area measuring device comprises in addition to the first laminate on the paddle which therefore always is present, a second laminate of an electrically low resistance material sandwiched between two layers of an electrically insulating material, which second laminate is provided on a side of the x-ray detector cover facing the breast with one of the layers of insulating material provided against the x-ray detector cover. In this manner both the upper and lower contact areas with the paddle and the x-ray detector cover, respectively can be measured. According to US 2014/341338 (and EP 2 819 581) this is however not a requirement, one can do also with only the first laminate provided on the paddle.

Relatively high costs are incurred with the apparatus according to US 2014/341338 (and EP 2 819 581) due to the fact that the known mammography-apparatus is embodied with interchangeable paddles that each have to be provided with a laminate to make the contact area measurement between the breast and the apparatus possible. The instant invention is aimed at reducing the costs of the apparatus according to US 2014/341338 (and EP 2 819 581) without sacrificing the benefits that it provides in terms of reliable and accurate measurement of the contact area between the mammography-apparatus and the breast.

### BRIEF SUMMARY OF THE INVENTION

The invention is embodied in the mammography-apparatus and in the method of measuring the contact area between the breast and the mammography-apparatus as specified in the appended claims.

In a first aspect of the invention the contact area measuring device is only provided on or incorporated in the detector cover facing the breast and the paddle is or paddles are embodied without a contact area measuring device. Within the scope of this invention the term “detector cover” embraces and includes an electrical insulation layer which is provided immediately on the detector and which separates the contact area measuring device from the bare detector. It is therefore not necessarily a part of the device which during use is in contact with the breast.

The invention is based on breaking with the prejudice that the contact area measurement should always at least be carried out regarding the contact area between the breast and the paddle. The inventors have however found that this is not necessary and that by taking appropriate measures it is also possible to only detect the contact area between the breast and the detector cover, without sacrificing the reliability and accuracy of the measurement. The invention brings the advantage to save costs since the replaceable paddles no longer require the application of a laminate as taught by EP 2 819 581. On the contrary with the invention it is only required to apply a single suitable laminate on the detector cover or to incorporate such laminate in the detector cover.

Suitably the contact area measuring device comprises a laminate of an electrically low resistance material sandwiched between two layers of an electrically insulating material, which laminate is provided on a side of the x-ray detector cover facing the breast with one of the layers of insulating material provided against the x-ray detector cover. It is possible that the laminate for application on the x-ray detector cover comprises a layer of an electrically low resistance material and only one layer of an electrically insulating material, and that a separate electrically insulating material is applied between the laminate and the x-ray detector cover. Within the terms of this application both embodiments are considered to constitute a laminate of an electrically low resistance material sandwiched between two layers of an electrically insulating material.

If such a laminate is provided on the side of the x-ray detector cover facing the breast, the measurement of the contact area between the breast and the x-ray detector cover is then preferably carried out by connecting the x-ray detector cover and the electrically low resistance material of the laminate on the x-ray detector cover to a measurement circuit which is powered by an alternating current excitation source, and that with reference to the potential of the x-ray detector cover the electrical potential of the electrically low resistance material of the laminate on the x-ray detector cover is measured, which electrical potential is then used as measure for the contact area between the breast and the x-ray detector cover.

The measurement of the contact area between the breast and the x-ray detector cover will then be based on the change of capacitance between the x-ray detector cover and the electrically low resistance material of the laminate on the x-ray detector cover, as this is determined by the breast placed between the paddle and the x-ray detector cover.

In all embodiments the laminate on the x-ray detector cover is preferably homogenous and substantially x-ray transparent.

The laminate on the x-ray detector cover preferably comprises materials with a low level of distortion or attenuation of the x-rays that are required for imaging the breast. On the other hand the insulating material of the laminate must suit the purpose of enabling the capacitance measurement according to the method of the invention. Suitably therefore both objectives can be met when the insulating material of the laminate is a thermoplastic having a relative dielectric constant of at least 1.8. Appropriately the insulating material of the laminate is then selected from the group comprising polyethylene, cyanoacrylate, polycarbonate. The dielectric constants of these details are 2 (polyethylene); 3 (cyanoacrylate); and 4.4 (polycarbonate). The laminate must be insulated from the conducting x-ray detector cover. For this purpose the laminate is preferably completed with an electrically insulating material on the side which will be provided to the detector cover. In this construction the electrically low resistance material of the laminate is sandwiched between two insulating layers.

Suitably the electrically low resistance material of the laminate is selected from the group comprising an electron-doped semiconductor and graphene.

Another preferable material to use for the electrically low resistance material of the laminate is silver nanowire. This silver nanowire preferably has a thickness of much less than 1 μm.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention.

FIG. 1 illustrates a mammography apparatus according to one embodiment of the present invention.

The invention will hereinafter be further elucidated with reference to the drawing providing a schematic figure of an embodiment of the mammography-apparatus according to the invention, which is not limiting to the appended claims.

### DETAILED DESCRIPTION OF THE INVENTION

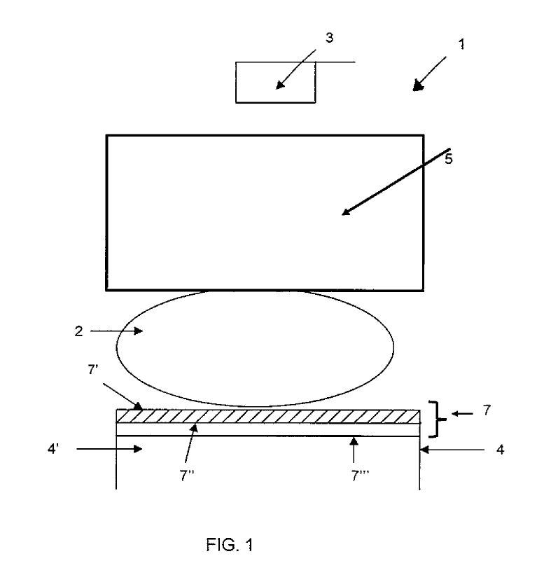

In the drawing FIG. 1 shows the mammography-apparatus of the invention, which is used for detecting malignant cells in a breast 2, wherein the x-ray detector is provided with a laminate.

The mammography-apparatus 1 comprises in known way an x-ray source 3 and an x-ray detector 4′ that cooperates with the x-ray source 3 for providing an x-ray image of the breast 2. The apparatus further has a paddle 5 for flattening the breast 2 by pressing the breast against the x-ray detector cover 4.

According to the invention there is a contact area measuring device for measuring a contact area between the breast 2 and the detector cover 4, which is embodied with a laminate 7 of an electrically insulating material and an electrically low resistance material.

In the shown embodiment the contact area measuring device comprises a laminate 7 of an electrically low resistance material 7″ sandwiched between two layers 7′, 7′″ of electrically insulating materials, which laminate 7 is provided on a side of the x-ray detector cover 4 facing the breast 2. The insulating material 7′ ensures isolation of the breast 2 from the electrically low resistance material 7″ of the laminate 7. In the shown example the laminate 7 is provided on the x-ray detector cover 4 wherein the intermediate insulating material 7′″ attached to the x-ray detector cover 4 forms an integral part of the laminate 7. It is however also possible that this latter intermediate insulating material 7′″ is applied separately from the laminate 7.

Preferably the insulating material 7′ of the laminate 7 is a thermoplastic having a relative dielectric constant of at least 1.8. The insulating material 7′ of the laminate 7 is preferably selected from the group comprising polyethylene, cyanoacrylate, polycarbonate. Further the insulating material 7′ of the laminate 7 has a thickness in the range 0.1-0.25 millimeters, preferably approximately 0.17 mm.

The electrically low resistance material 7″ of the laminate 7 preferably has a specific resistivity of less than 5×10⁻⁶ Ohm.m, and is selected from the group comprising an electron-doped semi-conductor and graphene. It is preferred however that the electrically low resistance material 7″ is silver nanowire, having a thickness of much less than 1 μm.

The mammography-apparatus 1 that is shown in FIG. 1 is used as follows. The contact area between the breast 2 and the x-ray detector cover 4 is measured by connecting x-ray detector cover 4 and the electrically low resistance material 7″ of the laminate 7 on the x-ray detector cover 4 to a measurement circuit which is powered by an alternating current excitation source. Then with reference to the potential of the x-ray detector cover 4, the electrical potential is measured of the electrically low resistance material 7″ of the laminate 7 on the x-ray detector cover 4, and this electrical potential is used as a measure for the contact area between the breast 2 and the x-ray detector cover 4.

The way in which the measurement circuit can be implemented can be in the form of applying voltage dividers or by measuring the time it takes to fully charge the capacitance between the low electrical resistivity material 7″ of the laminate 7 and the x-ray detector cover 4. The way this can be implemented is known to the person skilled in the art and requires no further elucidation with reference to the drawing.

It is explicitly remarked that the above elucidation of the features of the invention are not intended to limit the appended claims to the specific examples that are provided herewith. On the contrary, the invention is solely defined by the appended claims and the above elucidation merely serves to elucidate the claims if any ambiguity would reside in these claims. Without departing from the scope of the claims many variations are therefore feasible.

Although the invention has been described in detail with particular reference to these embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference

What is claimed is:

1. A mammography-apparatus for detecting malignant cells in a breast comprising:
an x-ray source and
an x-ray detector that cooperates with the x-ray source for providing an x-ray image of the breast and further comprising:
a paddle for flattening the breast by pressing it against an x-ray detector cover and comprising:
a contact area measuring device for measuring a contact area between the breast and the mammography-apparatus wherein the contact area measuring device comprises a laminate of an electrically low resistance material sandwiched between two layers of an electrically insulating material characterized in that the paddle is embodied without a contact area measuring device and that the contact area measuring device is only provided on or incorporated in the detector cover facing the breast.

2. The mammography-apparatus according to claim 1, characterized in that the laminate is provided on a side of the x-ray detector cover facing the breast with one of the layers of insulating material provided against the x-ray detector cover and that said contact area measuring device is embodied with a measurement circuit and equipped to be connected during use to an alternating current excitation source for measuring the electrical potential between the x-ray detector and the electrically low resistance material of the laminate on the detector.

3. The mammography-apparatus according to claim 1, characterized in that the laminate is homogenous and substantially x-ray transparent.

4. The mammography-apparatus according to claim 1, characterized in that the insulating material of the laminate is a thermoplastic having a relative dielectric constant of at least 1.8.

5. The mammography-apparatus according to claim 1, characterized in that the insulating material of the laminate is selected from the group comprising polyethylene, cyanoacrylate, polycarbonate.

6. The mammography-apparatus according to claim 1, characterized in that the insulating material of the laminate has a thickness in the range 0.1-0.25 millimeters, preferably approximately 0.17 mm.

7. The mammography-apparatus according to claim 1, characterized in that the electrically low resistance material of the laminate has a specific resistivity of less than 5×10⁻⁶ Ohm.m.

8. The mammography-apparatus according to claim 1, characterized in that the electrically low resistance material of the laminate is selected from the group comprising an electron-doped semiconductor and graphene.

9. The mammography-apparatus according to claim 1, characterized in that the electrically low resistance material of the laminate is silver nanowire.

10. The mammography-apparatus according to claim 9, characterized in that the silver nanowire has a thickness of less than 1 μm.
